(19) United States
(12) Patent Application Publication
(10) Pub. No.: US 2009/0326444 A1
(43) Pub. Date: Dec. 31, 2009

(54) DOUBLE-SIDED ADHESIVE SHEET

(75) Inventor: Masahiro Sakakibara, Tokyo (JP)

Correspondence Address: KNOBBE MARTENS OLSON & BEAR LLP, 2040 MAIN STREET, FOURTEENTH FLOOR, IRVINE, CA, 92614, US

(73) Assignee: NEMOTO KYORINDO CO., LTD., Tokyo (JP)

(21) Appl. No.: 12/090,807

(22) PCT Filed: Oct. 19, 2006
(86) PCT No.: PCT/JP2006/320831

§ 371 Date: Apr. 18, 2008

(30) Foreign Application Priority Data
Oct. 19, 2005 (JP) 2005-305062

(51) Int. Cl. A61M 5/145; B32B 33/00; A61B 5/00

(52) U.S. Cl. 604/67; 428/40.1; 428/42.1; 600/300

(57) ABSTRACT

The double-sided adhesive sheet (500) includes first, second, third and fourth peel sheets (510,520,530,540), and a sheet body 501. Each peel sheet has first, second, third and fourth affixing portion (511,521,531,541) affixed to the surface of the sheet body, and first, second, third and fourth peeling portion (512,522,532,542) not affixed to said surface. A boundary between the first affixing portion and the second affixing portion on the upper surface intersects a boundary between the third affixing portion and the fourth affixing portion on the lower surface. Owing to this structure, the peel sheets can be easily peeled off from the body.

### TECHNICAL FIELD

The present invention relates to a double-sided adhesive sheet including an adhesive sheet body having adhesion on both sides thereof, and more particularly, to a double-sided adhesive sheet for use in holding an injection needle in a human body and in placing a leak-detecting unit on the human body.

### BACKGROUND ART

Presently available medical apparatuses for capturing diagnostic images of patients include CT (Computed Tomography) scanners, MRI (Magnetic Resonance Imaging) apparatuses, PET (Positron Emission Tomography) apparatuses, ultrasonic diagnostic apparatuses and the like. Angiography apparatuses, MRA (MR angiography) apparatuses and the like are currently used as medical apparatuses for capturing vascular images of patients.

When the abovementioned medical apparatuses are used, a liquid such as a contrast medium or physiological saline may be injected into a patient. Chemical liquid injectors for automatically performing the injection have been put into practical use. A liquid syringe, for example including a cylinder member and a piston member slidably inserted into the cylinder member, is mounted on the injector. A syringe driving mechanism presses the piston member into the cylinder member. The cylinder member is filled with a liquid, and connected to a blood vessel of a human body near the surface thereof through an extension tube and an injection needle. The liquid in the liquid syringe is thus injected into the blood vessel of the human body by the chemical liquid injector.

The injector automatically injects the liquid at high pressure. For example, if the injection needle comes off and the liquid leaks inside the skin, an operator does not easily recognize it immediately. To solve this problem, various leak-detecting apparatuses have been proposed for detecting a leak of a liquid injected into a blood vessel of a human body near the surface thereof through an injection needle (see, for example, Patent Document 1 below).

The present applicants have proposed a leak-detecting system in which a leak-detecting apparatus as described above includes a leak-detecting unit and a detection apparatus body as separate components which wirelessly communicate with each other (see, for example, Patent Document 2: Japanese Patent Laid-Open No. 2005-152577 (US200410225255A1). In this case, since the leak-detecting unit can be reduced in size, it can be readily placed on a human body.

In Patent Document 2, the present applicants also have proposed that the leak-detecting unit should be attached to a human body with an sheet body of a double-sided adhesive sheet. A double-sided adhesive sheet of this type typically includes an sheet body which has a peel sheet affixed to each side thereof. The peel sheets are removed and a member is affixed to each of the exposed sides of the sheet body (see, for example, Patent Document 3).

Patent Document 1: Japanese Patent Laid-Open No. 2004-194802

Patent Document 2: Japanese Patent Laid-Open No. 2005-152577

Patent Document 3: Japanese Patent Laid-Open No. 10 (1998)-204387

However, since a typical double-sided adhesive sheet has an sheet body and peel sheets of the same shape, it is difficult to remove the peel sheet from the sheet body. Especially, the sheet body and the paired peel sheets are entirely flexible in a typical double-sided adhesive sheet. When an operator bends one of the peel sheets in order to remove that peel sheet from the sheet body, the sheet body and the other peel sheet may be bent together, to make the peeling difficult.

In use of such a double-sided adhesive sheet, the operator typically removes one of the peel sheets affixed to both sides of the sheet body and affixes the exposed adhesive surface of the sheet body to a first member. With this state, the operator removes the other peel sheet from the sheet body, and affixes a second member to the exposed adhesive surface. However, after the sheet is removed from the sheet body as described above, it is difficult for the operator to handle the sheet body so that his hand or finger does not touch the exposed adhesive surface.

If his hand or finger touches the adhesive surface of the sheet body, the adhesion thereof is deteriorated and the adhesive surface is soiled. The soiling of the adhesive surface of the sheet body may reduce the accuracy of detection by a leak-detecting unit attached to a human body with the double-sided adhesive sheet.

For example, the double-sided adhesive sheet described in Patent Document 3 includes peel sheets larger than the sheet body such that the peel sheets extend off the outer edge of the sheet body. This facilitates removal of the peel sheets from the sheet body and handling of the sheet body with no touch of any hand or finger on the exposed adhesive surface, after one of the peel sheets is removed from the sheet body. However, this results in the entire double-sided adhesive sheet larger than the effective sheet body, which causes problems in inventory and handling.

In actual use of the double-sided adhesive sheet described in Patent Document 3, an operator holds the pair of peel sheets with his right and left hands and then puts them apart for removing one of the peel sheets from the sheet body. However, it is indeterminate which sheet will be removed first from the sheet body. Therefore even if the operator may intend to remove the peel sheet held with his left hand and to affix the sheet body and the peel sheet held with his right hand to a member, unexpectedly the peel sheet held with his right hand may be removed first.

### DISCLOSURE OF THE INVENTION

The present invention has been made in view of the above-mentioned problems, and it is an object thereof to provide a double-sided adhesive sheet in which the peel sheets can be easily removed from an sheet body, and a peel sheet can be removed from a desired one of surfaces of the sheet body, and the sheet body can be handled readily after one peel sheet is removed therefrom, and the entire sheet is not larger than the entire sheet body.

The double-sided adhesive sheet according to the present invention includes an sheet body, a first peel sheet, a second peel sheet, a third peel sheet, and a fourth peel sheet. The sheet body has adhesion on each of an upper surface and a lower surface. The first peel sheet has a first affixing portion affixed to a portion of the upper surface of the sheet body, and a first peeling portion not affixed to the upper surface. The second peel sheet has a second affixing portion affixed to a portion of the upper surface of the sheet body, the first affixing portion being not affixed to the portion, and a second peeling portion not affixed to the upper surface, and placed at least partially on the first peeling portion. The third peel sheet has a third affixing portion affixed to a portion of the lower surface of the sheet body, and a third peeling portion not affixed to the lower surface. The fourth peel sheet has a fourth affixing portion affixed to a portion of the lower surface of the sheet body, the third affixing portion being not affixed to the portion, and a fourth peeling portion not affixed to the lower surface and placed at least partially on the third peeling portion. The boundary between the first affixing portion and the second affixing portion on the upper surface intersects the boundary between the third affixing portion and the fourth affixing portion on the lower surface. For example, while holding the first and second peeling portions with right hand, an operator detaches the third peeling portion and the fourth peeling portion in turn with his left hand from the sheet body to strip the third and fourth peel sheets from the sheet body. The third and fourth affixing portions are bent and stripped from the sheet body, but the first and second peeling portions placed on the upper surface of the sheet body are not bent downward, therefore the sheet body is not bent together with the third and fourth affixing portions. After the operator strips the third and fourth peel sheets from the sheet body, while holding the first and second peeling portions with his fingers as described above, he can affix the sheet body to a desired member while holding the first and second peeling portions with his fingers.

Various means referred to in the present invention may be arranged to perform their functions, and may comprise dedicated hardware for performing a predetermined function, a data processing apparatus whose predetermined function is given by a computer program, a predetermined function performed in a data processing apparatus according to a computer program, or a combination thereof.

Various components referred to in the present invention do not need to be separate entities. A plurality of components may be constructed as one member, a single component may be formed of a plurality of members, a certain component may be part of another component, or a certain component may have a portion overlapping a portion of another component.

Although upward and downward directions are specified in the present invention, these directions are defined for convenience to simply describe the relative relationship between components of the present invention and the definition does not limit any direction in manufacture or actual use when the present invention is implemented.

### EFFECT OF THE INVENTION

In the double-sided adhesive sheet according to the present invention, the boundary between the first affixing portion and the second affixing portion on the upper surface of the sheet body intersects the boundary between the third affixing portion and the fourth affixing portion on the lower surface. For example, while holding the first and second peeling portions with his right hand, the operator can detach the third peeling portion and the fourth peeling portion in turn with his left hand from the sheet body to easily strip the third and fourth peel sheets without removing the first and second peel sheets from the sheet body. When the third and fourth peel sheets are removed from the sheet body with the first and second affixing portions held with his right hand as described above, the operator can affix the sheet body to a member while holding the first and second peeling portions with his right hand. This achieves ease in handling of the sheet body with the peel sheet on one surface removed, and eliminates the need to form the peel sheet extending off the outer edge of the sheet body for the purpose of facilitating the handling and stripping. Consequently, an increased size of the entire sheet, which may cause inconvenience in inventory and handling, can be prevented.

### BRIEF DESCRIPTION OF THE DRAWINGS

FIG. 1 is an exploded perspective view showing a double-sided adhesive sheet according to an embodiment of the present invention;

FIGS. 2(a) and 2(b) are perspective views showing the double-sided adhesive sheet in use;

FIG. 3 is a perspective view showing how a leak-detecting unit is put on an arm of a human body with the double-sided adhesive sheet;

FIG. 4 is a schematic diagram showing the logical structure of a chemical liquid injector;

FIG. 5 is a block diagram showing the physical structure of an imaging diagnostic system;

FIG. 6 is a perspective view showing the outer appearance of the imaging diagnostic system;

FIG. 7 is a perspective view showing the outer appearance of the chemical liquid injector;

FIG. 8 is a perspective view showing how a liquid syringe is mounted on an injection head;

FIG. 9 is a longitudinal section showing the internal structure of the leak-detecting unit;

FIGS. 10(a) and 10(b) are graphs showing the detection results of rays in a predetermined wavelength band;

FIG. 11 is a flow chart showing the processing operation of the leak-detecting unit;

FIG. 12 is a flow chart showing the main routine in the processing operation of an injection apparatus body;

FIG. 13 is a flow chart showing the sub routine of the injection operation;

FIG. 14 is a longitudinal section showing the internal structure of a leak-detecting unit formed by way of trial in an experiment;

FIG. 15 is an exploded perspective view showing a double-sided adhesive sheet in a first modification;

FIG. 16 is an exploded perspective view showing a double-sided adhesive sheet in a second modification;

FIG. 17 is an exploded perspective view showing a double-sided adhesive sheet in a third modification;

FIG. 18 is a perspective view showing the double-sided adhesive sheet in the third modification;

FIG. 19 is an exploded perspective view showing a double-sided adhesive sheet in a fourth modification;

FIG. 20 is a perspective view showing first and second peel sheets of a double-sided adhesive sheet in a fifth modification;

FIG. 21 is a perspective view showing how a leak-detecting unit is put on an arm with a double-sided adhesive sheet in a sixth modification;

FIGS. 22(a) and 22(b) are perspective views showing how a leak-detecting unit is put on an arm with a double-sided adhesive sheet in a seventh modification; and

FIGS. 23(a) and 23(b) are perspective views showing modifications of the leak-detecting unit.

### DESCRIPTION OF REFERENCE NUMERALS

100 CHEMICAL LIQUID INJECTOR SERVING AS LEAK DETECTING SYSTEM
101 INJECTION APPARATUS BODY SERVING AS DETECTING APPARATUS BODY
146 INJECTION STOPPING MEANS
200 LIQUID SYRINGE
211 CYLINDER MEMBER
220 PISTON MEMBER
230 INJECTION NEEDLE SERVING AS OBJECT TO BE HELD
401, 421, 422, 600 LEAK DETECTING UNIT SERVING AS OBJECT TO BE AFFIXED
403 LIGHT-EMITTING DIODE CORRESPONDING TO LIGHT RAY EMITTING MEANS
404, 630 PHOTOTRANSISTOR CORRESPONDING TO LIGHT RAY DETECTING MEANS
500, 700, 710, 720, 740, 770 DOUBLE-SIDED ADHESIVE SHEET
501, 701, 760, 773 ADHESIVE SHEET BODY
510, 702, 721, 751, 772 FIRST PEEL SHEET
511, 716, 731 FIRST AFFIXING PORTION
512, 711, 726, 741 FIRST PEELING PORTION
520, 703, 722, 752 SECOND PEEL SHEET
521, 717, 732 SECOND AFFIXING PORTION
522, 712, 727, 742 SECOND PEELING PORTION
530, 704, 723 THIRD PEEL SHEET
531, 718, 733 THIRD AFFIXING PORTION
532, 713, 728, 743 THIRD PEELING PORTION
540, 705, 724 FOURTH PEEL SHEET
541, 719, 734 FOURTH AFFIXING PORTION
542, 714, 729, 744 FOURTH PEELING PORTION
620 INFRARED RAY LED CORRESPONDING TO LIGHT RAY EMITTING MEANS
761, 762, 771, 774 IMAGE
A ARM OF HUMAN BODY
V BLOOD VESSEL

### BEST MODE FOR CARRYING OUT THE INVENTION

### Configuration of Embodiment

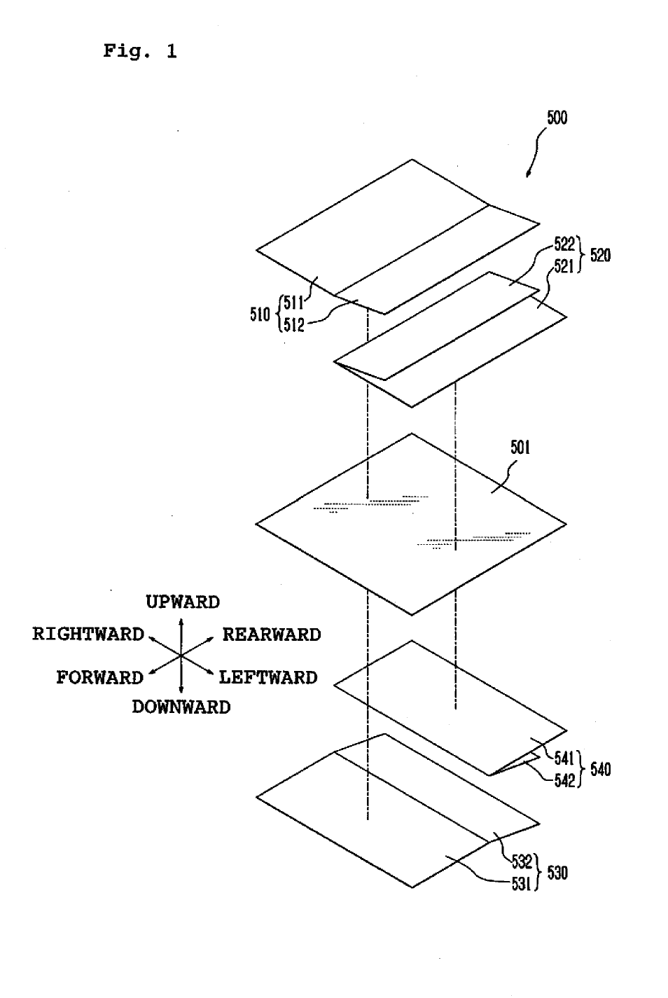

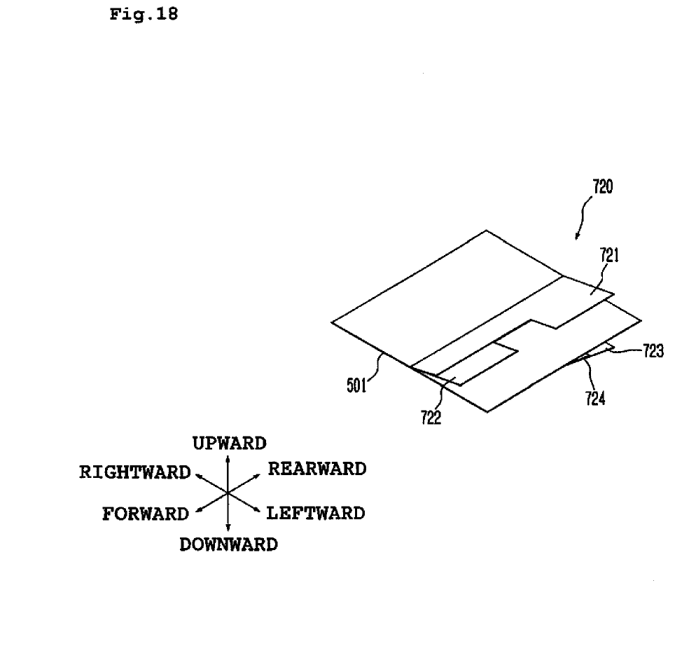

An embodiment of the present invention will hereinafter be described with reference to FIGS. 1 to 23. Although the directions of forward, rearward, leftward, rightward, upward, and downward are specified as shown in the embodiment, these directions are defined for convenience to facilitate the description, and the definition does not limit any direction in manufacture or actual use of the apparatus of the present invention.

As shown in FIGS. 4 to 6, imaging diagnostic system 1000 of the embodiment according to the present invention comprises chemical liquid injector 100, liquid syringe 200, and CT scanner 300 serving as an imaging diagnostic apparatus. Although described later in detail, chemical liquid injector 100 of the embodiment is formed integrally with a leak-detecting system and includes disposable double-sided adhesive sheet 500 as part of the system as shown in FIGS. 1 to 3.

Double-sided adhesive sheet 500 of the embodiment is formed of sheet body 501, first peel sheet 510, second peel sheet 520, third peel sheet 530, and fourth peel sheet 540. Adhesive sheet body 501 has adhesion on each of an upper surface and a lower surface. Adhesive sheet body 501 is formed of, for example, a flexible resin sheet of a square of 5 cm×5 cm. An adhesive is applied to both sides of the resin sheet. The resin sheet and the adhesive may be clear and colorless, but they may be clear and colored, or may be opaque as long as they favorably transmit rays in a wavelength band described later.

Each of first, second, third, and fourth peel sheets 510, 520, 530, and 540 is formed of, for example, a flexible paper sheet of a rectangle of 5 cm×4 cm. A resin layer having a high peeling property is formed integrally with one side of each peel sheet that is affixed to sheet body 501. Each of peel sheets 510, 520, 530, and 540 is bent along its longitudinal direction to provide first, second, third, and fourth affixing portions 511, 521, 531, and 541 of a size of 5 cm×2.5 cm and first, second, third, and fourth peeling portions 512, 522, 532, and 542 of a size of 5 cm×1.5 cm, for example.

In double-sided adhesive sheet 500 of the embodiment, when sheet body 501 is positioned with its four sides toward the forward, rearward, leftward, and rightward directions, and its two surfaces toward the upward and downward directions, first affixing portion 511 of first peel sheet 510 is affixed to the left half of the upper surface of sheet body 501 and second affixing portion 521 of second peel sheet 520 is affixed to the right half of the upper surface.

Third affixing portion 531 of third peel sheet 530 is affixed to the forward half of the lower surface of sheet body 501 and fourth affixing portion 541 of fourth peel sheet 540 is affixed to the rearward half of the lower surface. Thus, a boundary between first and second affixing portions 511 and 521 is orthogonal to a boundary between third and fourth affixing portions 531 and 541.

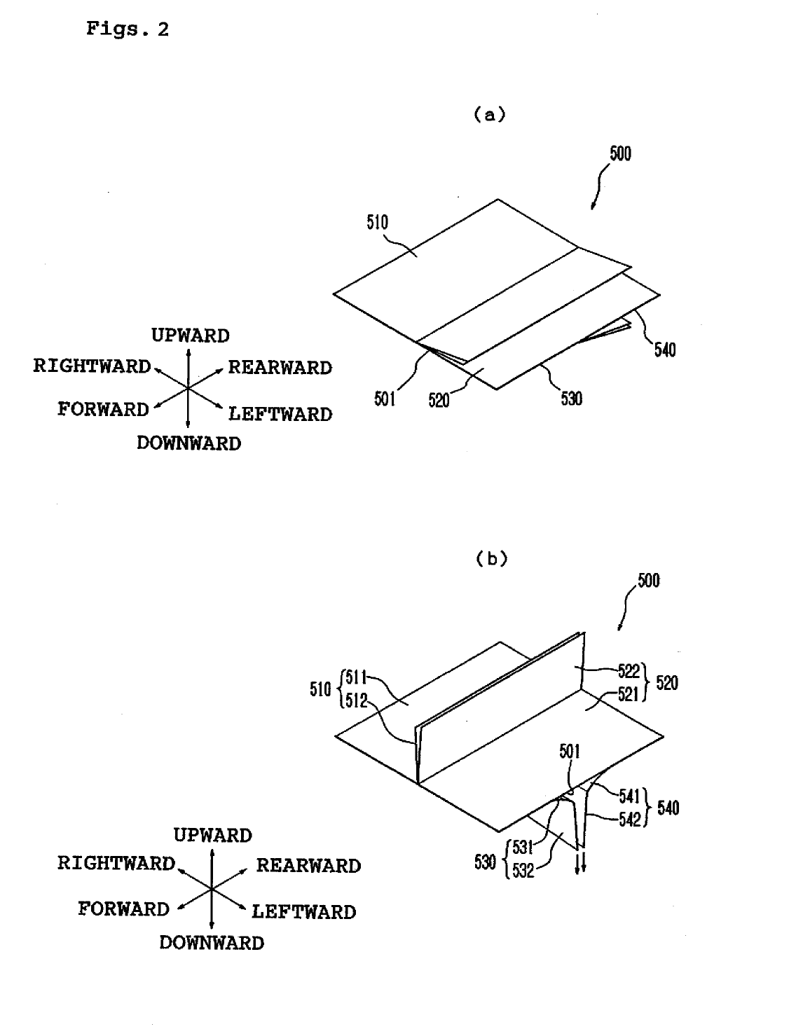

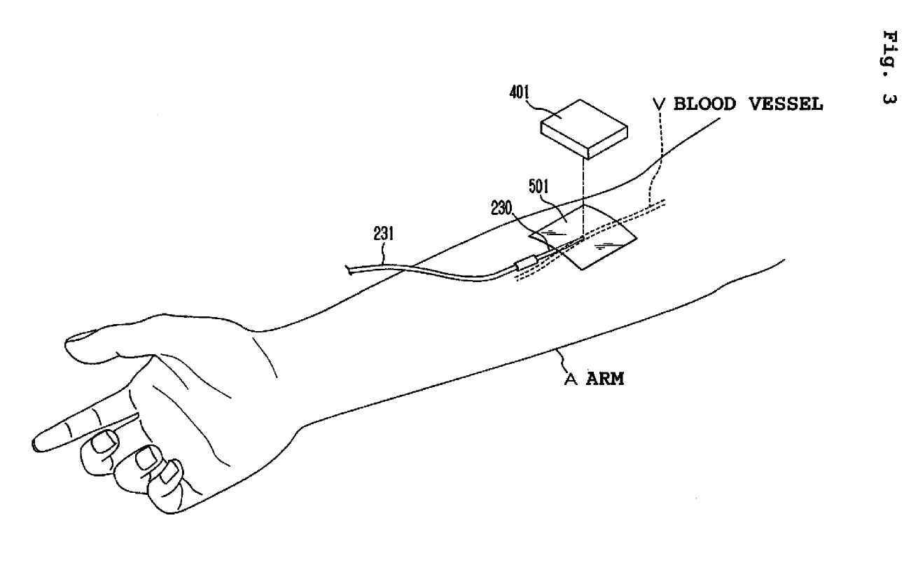

Since first, second, third, and fourth peel sheets 510, 520, 530, and 540 are flexible as described above, first, second, third, and fourth peeling portions 512, 522, 532, and 542 are placed flatly one on another over second and fourth affixing portions 521 and 541 as shown in FIGS. 1 and 2(a) when sheet 500 is stocked, while peeling portions 512 to 542 are erected in use as shown in FIG. 2(b).

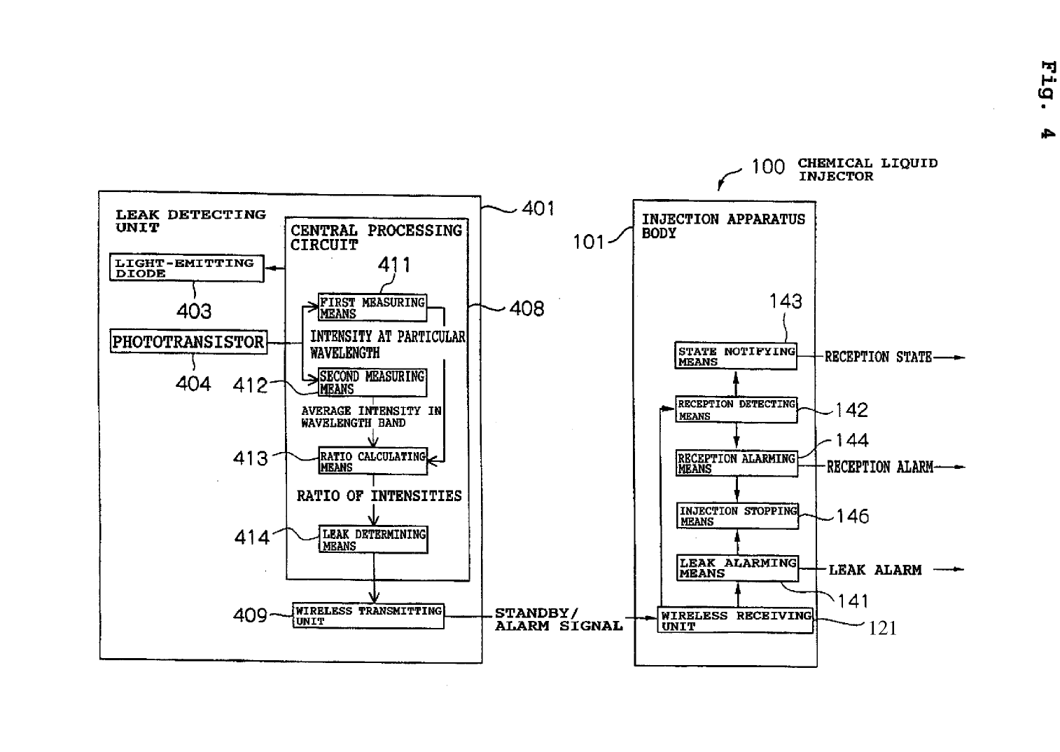

As shown in FIG. 3, double-sided adhesive sheet 500 of the abovementioned structure is used for placing a leak-detecting unit 401 of chemical liquid injector 100 of the embodiment on arm A of a human body, and is used for holding an injection needle 230 which is inserted into blood vessel V of arm A. Injection needle 230 is connected through extension tube 231 to liquid syringe 200 which is driven by chemical liquid injector 100.

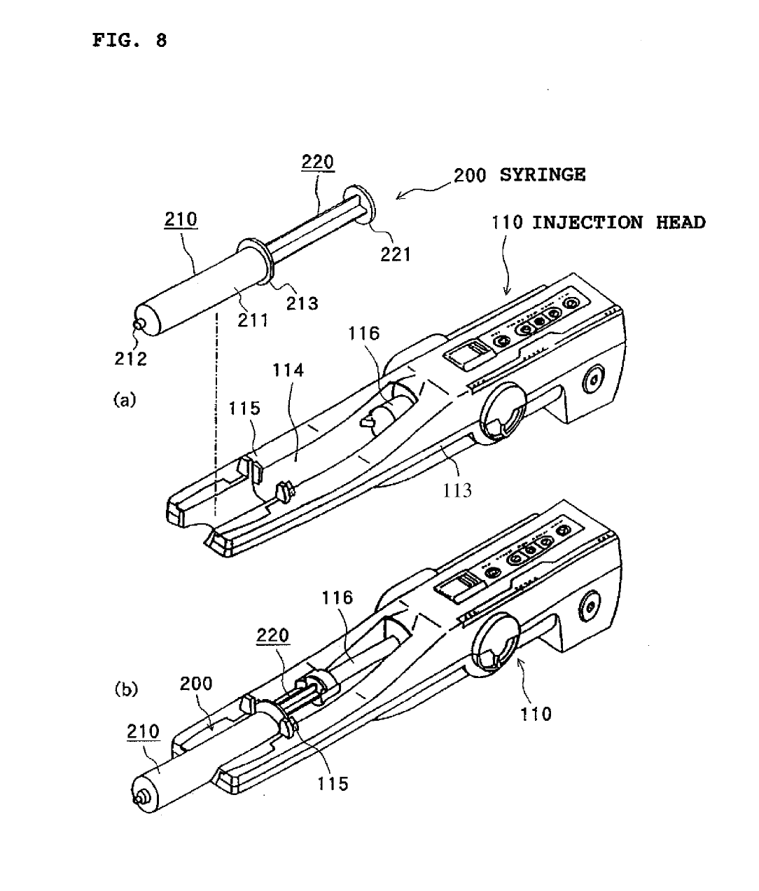

As shown in FIG. 8, the liquid syringe 200 comprises a cylinder member 210 and a piston member 220 wherein the piston member 220 is slidably inserted into the cylinder member 210. The cylinder member 210 includes cylindrical hollow body 211 which has a conduit 212 formed at the closed leading end surface.

The trailing end of the body 211 of cylinder member 210 is opened and the piston member 220 is inserted from the opening into the interior of the body 211. The cylinder member 210 has a cylinder flange 213 formed in the outer circumference of the trailing end. The piston member 220 has a piston flange 221 formed in the outer circumference of the trailing end.

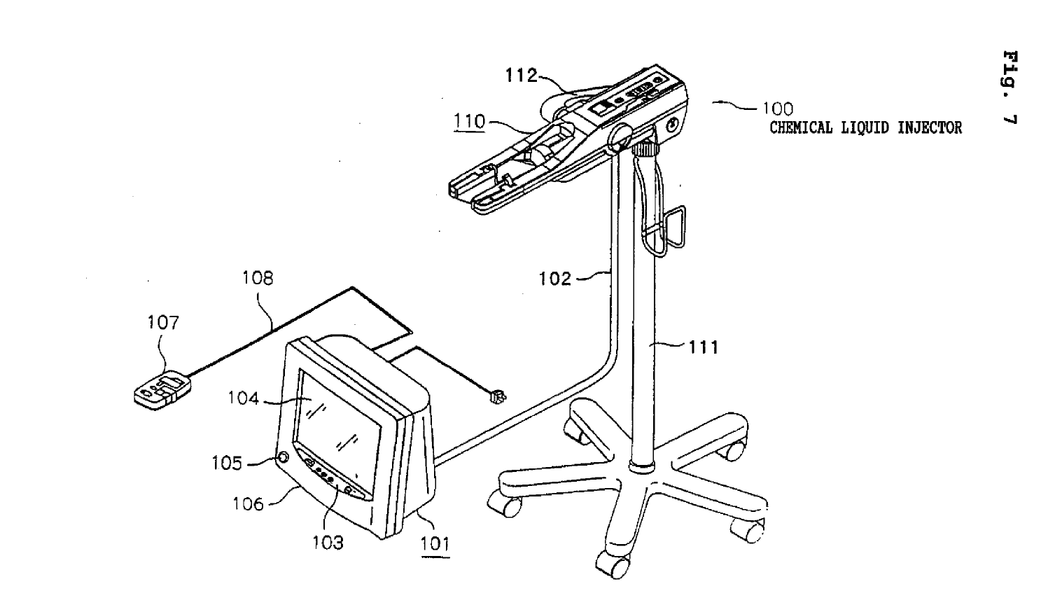

As shown in FIG. 7, chemical liquid injector 100 of the embodiment has injection control unit 101 and injection head 110 constructed as separate components which are wire-connected through communication cable 102. Injection head 110 is attached to the top end of caster stand 111 by movable arm 112.

As shown in FIG. 8, head body 113 of injection head 110 has concave portion 114 formed in its upper surface in a semi-cylindrical shape which fits cylinder member 210 of liquid syringe 200. Cylinder holding mechanism 115 is formed in the forward section of concave portion 114 for removably holding cylinder flange 211 of liquid syringe 200.

Piston driving mechanism 116 is placed in the rearward section of concave portion 114 of injection head 110, for holding and sliding piston flange 221. Piston driving mechanism 116 removably holds and slides piston member 220 of liquid syringe 200 in the forward and rearward directions.

In chemical liquid injector 100 of the embodiment, as shown in FIG. 3, liquid syringe 200 held in injection head 110 is connected to blood vessel V of arm A of a human body, through extension tube 231 and injection needle 230, for example. Injection needle 230 is held by sheet body 501.

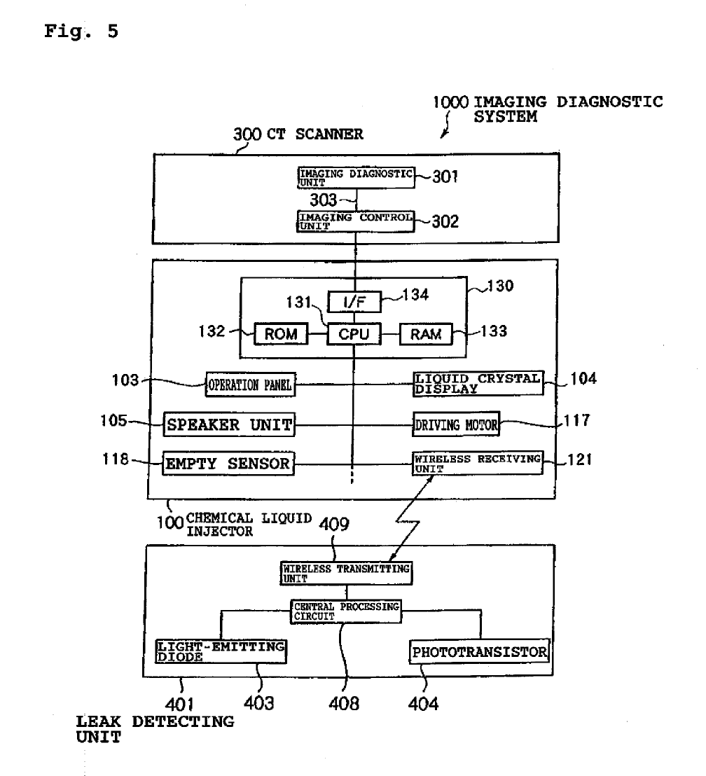

As shown in FIG. 5, piston driving mechanism 116 has driving motor 117 including an ultrasonic motor or the like as a driving source which is free from generation of magnetic field even in operation, and slides piston member 220 through a screw mechanism (not shown) or the like. Empty sensor 118 is contained in piston driving mechanism 116, the sensor detects the position of piston flange 221, to sense the completion of liquid injection with liquid syringe 200.

Injection control unit 101 connected through communication cable 102 to injection head 110 formed as described above contains a computer unit 130 and is wire-connected to imaging control unit 302 of CT scanner 300 through communication network 304.

As shown in FIG. 7, injection control unit 101 has operation panel 103, liquid crystal display 104 serving as a data display means, speaker unit 105 and the like, all of which are disposed on the front face of unit housing 106. Injection control unit 101 is wire-connected to controller unit 107 as a separate component through connector 108.

Chemical liquid injector 100 of the embodiment is integral with the leak-detecting system, and injection control unit 101 serves as the detecting apparatus body. Leak-detecting unit 401 is formed as a separate component from injection control unit 101 and wirelessly communicates with injection control unit 101.

The injection control unit 101 also contains a wireless receiving unit 121 serving as a wireless receiving means, which wirelessly communicates with leak-detecting unit 401. As shown in FIG. 5, various devices such as wireless receiving unit 121 are wire-connected to computer unit 130.

Computer unit 130 is formed of a so-called one-chip microcomputer provided with hardware such as CPU (Central Processing Unit) 131, ROM (Read Only Memory) 132, RAM (Random Access Memory) 133, I/F (Interface) 134 and the like. Computer unit 130 has an appropriate computer program installed as firmware or the like on an information storage medium such as ROM 132. CPU 131 executes various types of processing in accordance with the computer program.

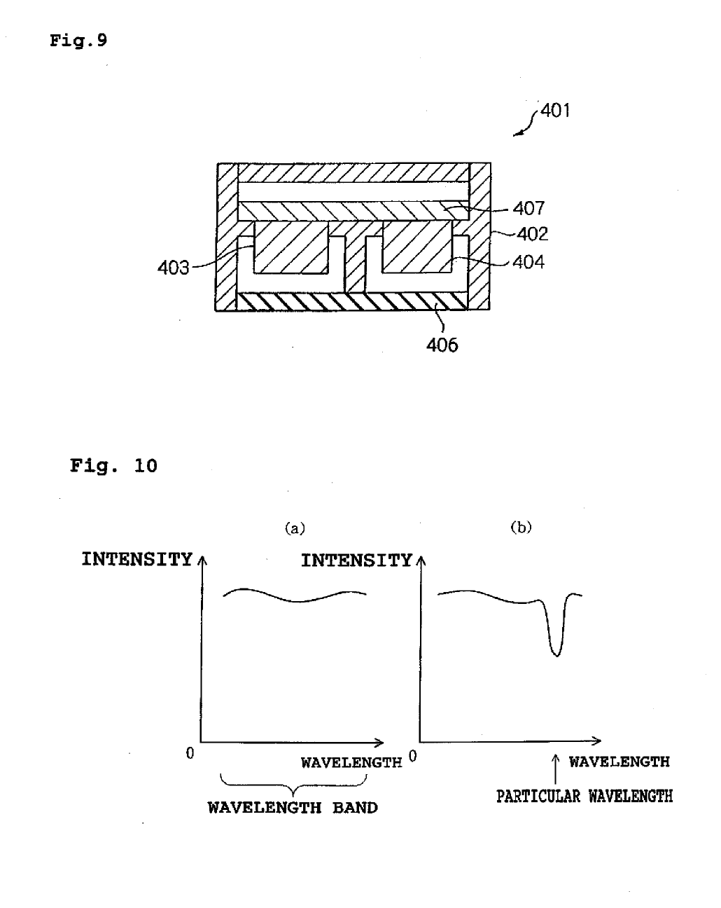

The Leak-detecting unit 401 has a unit housing 402 in a box shape as shown in FIG. 9. The unit housing 402 contains a light-emitting diode 403 serving as a ray emitting means, and a phototransistor 404 serving as a ray detecting means such that they face downward.

The light-emitting diode 403 emits rays in a predetermined wavelength band in a downward direction. The photodiode 404 receives the rays in the wavelength band from below. More specifically, the light-emitting diode 403 emits the rays in the wavelength band which are reflected uniformly by the internal tissues of arm A. The wavelength band of the rays includes a particular wavelength, at which the rays are absorbed at a high rate by a liquid such as a contrast medium, the reflectivity for a liquid such as a contrast medium is lower than the reflectivity for the internal tissues of arm A.

When the liquid is not leaked inside the arm A, the strength of the rays, in the wavelength band, reflected inside arm A and then detected, is substantially constant as shown in FIG. 10(a). Whereas, if the liquid is leaked inside arm A, the strength of the rays reflected inside arm A and then detected is extremely reduced at the particular wavelength as shown in FIG. 10(b).

An optical filter 406 is placed below (and opposite to) the light-emitting diode 403 and the phototransistor 404. The optical filter 406 allows only the rays in the abovementioned wavelength band to pass therethrough. A circuit board 407 is placed in an upper portion in the leak-detecting unit 401. As shown in FIG. 5, the circuit board comprises light-emitting diode 403, phototransistor 404, central processing unit 408, and wireless transmitting unit 409 serving as a wireless transmitting means are implemented on.

The central processing unit 408 is wire-connected to the light-emitting diode 403, phototransistor 404, and wireless transmitting unit 409. It causes light-emitting diode 403 to continuously emit rays, and continuously takes the output from phototransistor 404. Central processing unit 408 is formed as a logical circuit of a predetermined structure and has various types of hardware, functioning as a first measuring means 411, a second measuring means 412, a ratio calculating means 413, and a leak determining means 414.

The first measuring means 411 is formed of, for example, an A/D (Analog/Digital) converter and an arithmetic processing circuit (not shown), and it measures the intensity of the rays in the particular wavelength detected by phototransistor 404. The second measuring means 412 is formed of an A/D converter, an arithmetic processing circuit and the like, and it measures the average intensity of the detected rays in the wavelength band.

The intensity of rays in the particular wavelength, measured as described above, may be an intensity of rays only at the particular wavelength. Alternatively, the intensity of rays in the particular wavelength may be the average intensity of rays in outside of predetermined range of wavelengths that includes the particular wavelength at the center, for example. The average intensity of rays in the wavelength band may be the average of rays in the entire wavelength band including the abovementioned particular wavelength, or, the average of rays except for a predetermined range including the particular wavelength at the center in the abovementioned wavelength band, for example.

The ratio calculating means 413 and the leak determining means 414 are formed of a predetermined arithmetic processing circuit and the like. The ratio calculating means 413 calculates the ratio of the intensity of rays at the particular wavelength to the average intensity of rays in the wavelength band measured as described above. The leak determining means 414 determines the occurrence of a leak, if the calculated ratio is lower than a predetermined reference value.

Central processing circuit 408 causes wireless transmitting unit 409 to continuously transmit a predetermined standby signal through radio waves when leak determining means 414 does not determine the occurrence of a leak as described above. If the occurrence of a leak is determined, central processing circuit 408 causes wireless transmitting unit 409 to wirelessly transmit a predetermined alarm signal.

In chemical liquid injector 100 of the embodiment, microprocessor 130 integrates and controls the respective components in accordance with the computer program as described above to logically have various means, as various functions, such as leak alarming means 141, reception detecting means 142, state notifying means 143, reception alarming means 144, and injection stopping means 146, as shown in FIG. 4.

The leak alarming means 141 corresponds to the function of microcomputer 130 which controls the operation of speaker unit 105 and liquid crystal display 104 in accordance with the computer program. The leak alarming means 141 outputs and notifies leak alarm with a sound output from speaker unit 105 and an image displayed on liquid crystal display 104, when standby signal wirelessly received by wireless receiving unit 121 is switched to alarm signal,

The reception detecting means 142 corresponds to the function of microprocessor 130, which detects the data about the operation state of wireless receiving unit 121, and it detects the reception state of a radio signal. The state notifying means 143 corresponds to the function of microprocessor 130 which controls the operation of liquid crystal display 104 and the like, and it outputs and notifies the reception state detected by reception detecting means 142 with an image displayed on liquid crystal display 104 or the like.

The reception alarming means 144 corresponds to the function of microprocessor 130 which controls the operation of speaker unit 105 and liquid crystal display 104, and it outputs and notifies a reception alarm with a sound output from speaker unit 105 and an image displayed on liquid crystal display 104, when the reception state detected by reception detecting means 142 is degraded below than a predetermined state. The injection stopping means 146 corresponds to the function of microprocessor 130 which controls the operation of driving motor 117 of piston driving mechanism 116 and the like, and it stops driving motor 117 to end the injection of the liquid when at least one of the leak alarm and the reception alarm is output.

Although the abovementioned various means 141 to 146 of chemical liquid injector 100 are accomplished by pieces of hardware such as speaker unit 105 as required, they are mainly implemented by microprocessor 130 functioning in accordance with the installed computer program.

Such a computer program is described to cause microprocessor 130 to perform processing operations including the output and notification of the leak alarm from speaker unit 105 and on liquid crystal display 104 when wireless receiving unit 121 wirelessly receives the alarm signal, the stop of driving motor 117 in response to the output of the leak alarm, the detection of the reception state of wireless receiving unit 121, the output and notification of the reception state on liquid crystal display 104, the output and notification of the reception alarm from speaker unit 105 and on liquid crystal display 104 when the reception state is degraded below the predetermined state, and the stop of driving motor 117 in response to the output of the reception alarm.

Since double-sided adhesive sheet 500 of the embodiment is used for placing leak-detecting unit 401 on arm A. The sheet body 501 comprises the resin sheet and the adhesive which favorably pass the abovementioned rays in the predetermined wavelength band emitted by light-emitting diode 403 and then received by phototransistor 404.

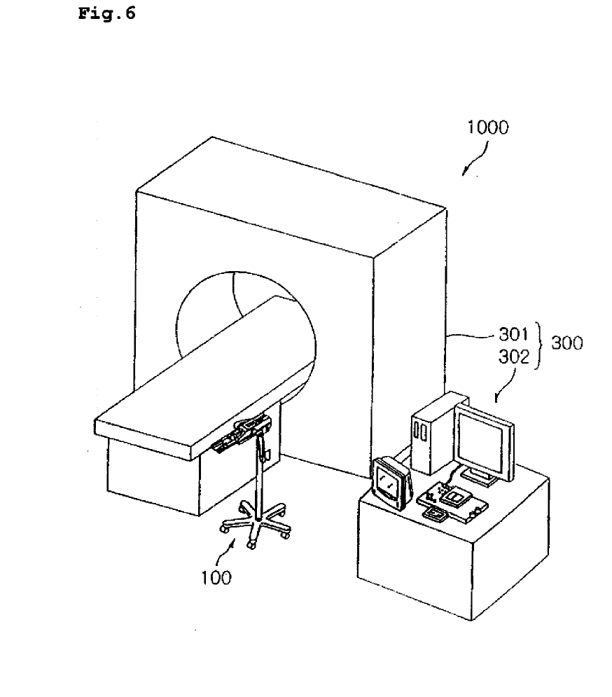

As shown in FIGS. 5 and 6, CT scanner 300 of imaging diagnostic system 1000 in the embodiment, includes an imaging diagnostic unit 301 serving as a mechanism for performing imaging, and an imaging control unit 302. The imaging diagnostic unit 301 and the imaging control unit 302 are wire-connected through communication network 303. The imaging diagnostic unit 301 shoots a diagnostic image of a patient (not shown). The imaging control unit 302 controls the operation of imaging diagnostic unit 301.

For simplify the representation, control unit 302 and the whole chemical liquid injector 100 are placed near imaging diagnostic unit 301 of CT scanner 300 in FIG. 6. However, for practical use in a medical facility, only injection head 110 is placed near imaging diagnostic unit 301, and control unit 302 and injection control unit 101 are disposed in a different room.

### Operation of the Embodiment

When the imaging diagnostic system 1000 of the embodiment is used in the abovementioned arrangement, for example, the operator connects the injection needle 230 through extension tube 231 to liquid syringe 200 filled with a liquid such as a contrast medium. And the injection needle 230 is inserted into blood vessel V of arm A of a patient in imaging diagnostic unit 301 of CT scanner 300 as shown in FIG. 3.

Next, the operator prepares disposable double-sided adhesive sheet 500. In a state for stock, first, second, third, and fourth peeling portions 512, 522, 532, and 542 are placed flatly one on another over second and fourth affixing portions 521 and 541 as shown in FIG. 2(a). The operator erects first to fourth peeling portions 512 to 542 with his fingers as shown in FIG. 2(b).

Then, for example, while holding first and second peeling portions 512 and 522 with his right hand, the operator separates the third peeling portion 532 and the fourth peeling portion 542 in turn, from sheet body 501 with his left hand, to remove the sheets 530 and 540 in turn from sheet body 501.

The third and forth affixing portions 531 and 541 are removed from sheet body 501 with bending. However, since the direction of the boundary between the third and fourth peel sheets 530 and 540 intersects the direction of the boundary between the first and second peeling portions 512 and 522, the sheet body 501 is not bent together with the third and fourth affixing portions 531 and 541.

More particularly, for example in stripping of the affixing portion 531 of third peel sheet 530 downward from the lower surface of the sheet body 501, the stripping portion is shifted from the center toward the front of the lower surface of sheet body 501, and accordingly, the position of the lower surface of sheet body 510 that is pulled downward is shifted from the center toward the front.

The first and second peel sheets 510 and 520 are affixed to the upper surface of sheet body 501 and the plane direction of first and second peeling portions 511 and 521 is in parallel with the forward and rearward directions and the upward and downward directions. Thus, the first and second peeling portions 511 and 521 which are not bent downward are located along the forward and rearward directions on the upper surface of sheet body 501, so that the sheet body 501 is not bent downward when the third and fourth affixing portions 531 and 541 are stripped downward as described above.

The operator removes third and fourth peel sheets 530 and 540 from sheet body 501 with his left hand while holding first and second peeling portions 512 and 522 with his right hand as described above. Then, the lower surface of the sheet body 510 is affixed upon the arm A, while the operator holds the first and second peeling portions 512 and 522 with his right hand.

The sheet body 501 is affixed to the position where injection needle 230 is inserted into the arm A, thereby the injection needle 230 is held. Then, the operator peels the first and second peel sheets 510 and 520 off the upper surface of sheet body 501 attached to arm A as described above. The leak-detecting unit 401 is then affixed onto the exposed the upper surface of sheet body 501. In this manner, the leak-detecting unit 401 is placed on the surface of the arm A through which injection needle 230 is inserted into blood vessel V.

Then, the liquid syringe 200 is mounted on the injection head 110 of chemical liquid injector 100. Each power switch (not shown) of leak-detecting unit 401 and injection control unit 101 is turned on. For example, an operation mode for using leak-detecting unit 401 is set on the injection control unit 101 through a predetermined operation.

Then, as shown in FIG. 11, the leak-detecting unit 401 emits rays, in the predetermined wavelength band from light-emitting diode 403, toward the position where the injection needle 230 is inserted into blood vessel V of arm A. The rays is detected by the phototransistor 404 (step S1). The intensity of the rays in the particular wavelength within the detected predetermined wavelength band is measured (step S2), and the average intensity of the rays in the wavelength band is measured (step S3). The ratio of the measured intensity of the rays at the particular wavelength to the measured average intensity of the rays in the wavelength band is calculated (step S4), and then the calculated ratio is compared with the predetermined reference value (step S5).

If the ratio is not lower than the reference value, the occurrence of a leak of the liquid is not determined. The leak-detecting unit 401 wirelessly transmits a standby signal representing “no leak” to injection control unit 101 (step S6). On the other hand, when the abovementioned ratio is lower than the reference value, the occurrence of a leak of the liquid is determined (step S7). The leak-detecting unit 401 wirelessly transmits an alarm signal representing “occurrence of leak” to the injection control unit 101 (step S8).

The method of detecting a leak in chemical liquid injector 100 of the embodiment will hereinafter be described in brief. The light-emitting diode 403 of leak-detecting unit 401 emits the rays in the wavelength band which are reflected uniformly by the internal tissues of arm A as described above. The wavelength band includes particular wavelength in which the reflectivity for a predetermined liquid such as a contrast medium is lower than the reflectivity for the internal tissues of arm A.

When the liquid is not leaked inside arm A, the rays, in the wavelength band reflected inside arm A and then detected, have a substantially constant intensity as shown in FIG. 10(a). However, if the liquid is leaked, the rays have an intensity which is extremely reduced only at the particular wavelength as shown in FIG. 10(b).

The ratio of the intensity at the particular wavelength to the average intensity in the wavelength band is approximately 1 when no liquid is leaked inside arm A, whereas the ratio is extremely reduced below 1 when the liquid is leaked. In the chemical liquid injector 100 of this embodiment, for example, the ratio may be compared with a reference value such as 0.8, in order to determine whether or not the liquid is leaked with high accuracy.

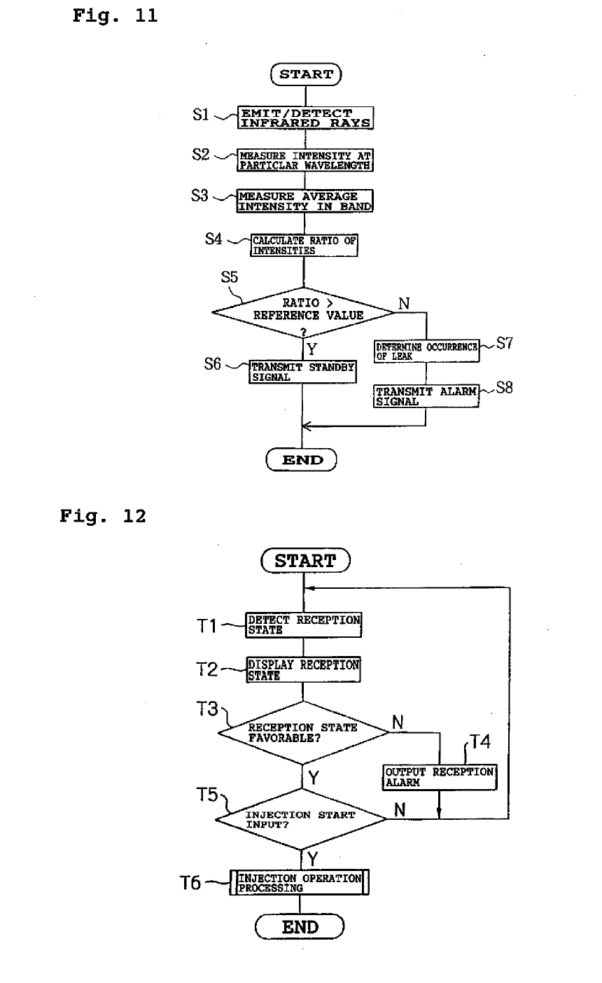

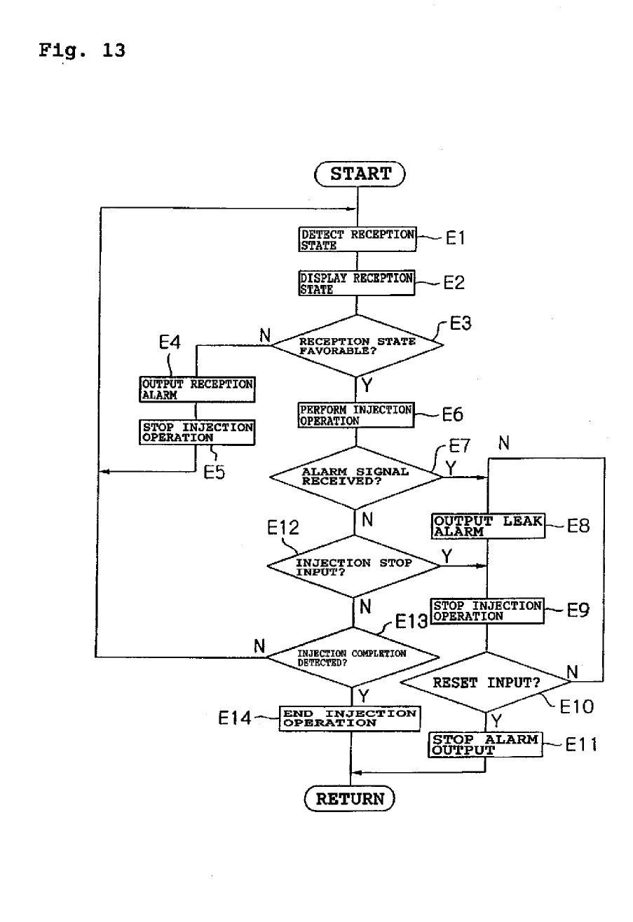

As shown in FIG. 12, the injection control unit 101 continuously detects the reception state of radio waves during the operation state in which the leak-detecting unit 401 is used (step T1). The unit 101 displays the reception state, in real time, for example as a bar graph on the liquid crystal display 104 (step T2).

With this operation, the operator can check the reception state of radio waves from leak-detecting unit 401 in real time while operating injection control unit 101. If the reception state is not favorable, the operator can adjust the position of the injection control unit 101 or the position of leak-detecting unit 401.

When the reception state is degraded below the predetermined state as described above (step T3), the injection control unit 101 outputs a reception alarm such as “Radio wave reception impossible. Check communication state” with an image on the liquid crystal display 104 and with a sound form speaker unit 105 (step T4).

Since the injection control unit 101 does not accept any entry operation to start injection, until the reception state from leak-detecting unit 401 is switched a favorable state (steps T3 to T5), the injection control unit 101 can start the injection operation of the liquid only when the reception state is favorable (step T6).

After the injection control unit 101 receives the entry operation to start injection of the liquid (steps T5 and T6), it continuously detects the reception state of radio waves, and outputs and displays it on liquid crystal display 104 in real time as shown in FIG. 13 (steps E1 and E2).

When the reception state is degraded below the predetermined state, the injection control unit 101 outputs and notifies the alarm on liquid crystal display 104 and from speaker unit 105 (steps E3 and E4), the injection is performed only when the reception state is favorable (steps E3 to E6).

During the injection control unit 101 performs the injection in the favorable reception state (step E6), if the wirelessly received standby signal is switched to the alarm signal (step E7), the unit outputs a leak alarm such as “Removal of injection needle detected. Check injection needle” with an image on liquid crystal display 104 and a sound from speaker unit 105 (step E8).

In this case, injection is stopped (step E9), there for liquid injection is not continued under a condition where the injection needle 230 comes off blood vessel V. The abovementioned leak alarm is continued until predetermined reset operation is input on injection control unit 101 (steps E10 and E11), so that the operator may recognize the leak alarm without fail.

In chemical liquid injector 100 of this embodiment, upon recognition of the leak alarm, the operator appropriately inserts injection needle 230 into blood vessel V and then makes entry operation to start the injection of the liquid on operation panel 103. In response thereto, the liquid injection can be started again (steps T5 and T6).

When the operator makes entry operation to stop the injection on operation panel 103 (step E12), the injection control unit 101 stops the injection of the liquid (step E9). When the empty sensor 118 senses completion of the injection of the liquid (step E13), injection control unit 101 ends the injection of the liquid (step E14).

### Effect of the Embodiment

In the imaging diagnostic system 1000 of the embodiment, when injection needle 230 is inserted into blood vessel V of the patient to inject the liquid as described above, the double-sided adhesive sheet 500 can hold injection needle 230 and place leak-detecting unit 401 on the surface of arm A, at the position where the injection needles 230 is inserted to blood vessel V.

In double-sided adhesive sheet 500 of the embodiment, the operator peels for example the third peeling portion 532 and fourth peeling portion 542, in turn, with his left hand from the sheet body 501 while holding the first and second peeling portions 512 and 522 with his right hand. It is easy to remove the third and fourth sheets 530 and 540 without removing the first and second sheets 510 and 520 from sheet body 501.

Specifically, since the direction of the boundary between third and fourth peel sheets 530 and 540 intersects the direction of the boundary between first and second peeling portions 512 and 522, the sheet body 501 is not bent together with the third and fourth affixing portions 531 and 541 to be stripped. As a result, the third and fourth peel sheets 530 and 540 and the like can be removed from the sheet body 501 extremely readily.

After the operator separates the third and fourth peel sheets 530 and 540 with his left hand from sheet body 501 while holding first and second peeling portions 512 and 522 with his right hand, he can affix the lower surface of sheet body 501 to arm A or the like, while holding the first and second peeling portions 512 and 522 with his right hand. It is easy to handle sheet body 501 with one of the adhesive surfaces exposed.

The operator's hand or finger does not touch the exposed adhesive surface of sheet body 501, thus the adhesive surface of sheet body 501 is not soiled. The preclusion of soiling of the adhesive surface of sheet body 501 can prevent deteriorated adhesion of sheet 500. Moreover, the soiling of the portion of arm A where the injection needle 230 is inserted is prevented, and the detection accuracy of leak-detecting unit 401 is assured. Particularly, in double-sided adhesive sheet 500 of the embodiment, the sheet body 501 favorably passes the rays in the predetermined wavelength band emitted and received by leak-detecting unit 401, so that sheet body 501 does not impair the detection accuracy of leak-detecting unit 401.

The double-sided adhesive sheet 500 of the embodiment has no need to form peel sheets 510, 520, 530, and 540 extending off the outer edge of sheet body 501 for greater ease in handling and stripping. This can avoid an increase in the overall size of sheet 500, which may cause inconvenience in inventory and handling.

As shown in FIG. 2(b), the peeling portions 512, 522, 532, and 542 are erected on the surface of the sheet body 501 to facilitate stripping of the peel sheets 510, 520, 530, and 540 and handling of sheet body 501. The peeling portions 512, 522, 532, and 542 can be placed one on another on the surface of sheet body 501 as shown in FIG. 2(a) to realize double-sided adhesive sheet 500 which, as a whole, has a sheet shape of the same size as sheet body 501, thereby allowing extreme ease in stock and handling. In addition, peel sheets 510, 520, 530, and 540 have the same shape to attain high productivity in double-sided adhesive sheet 500 of the embodiment.

In the chemical liquid injector 100 of the embodiment, light-emitting diode 403 emits the rays in the predetermined wavelength band including the particular wavelength at which the reflectivity for the liquid is lower than the reflectivity for the internal tissues within arm A as described above, and the occurrence of a leak is determined when the ratio of the intensity of the rays at the particular wavelength to the average intensity of the rays in the wavelength band reflected inside arm A is reduced below the reference value.

For example, when the intensity of rays reflected inside arm A changes due to variations in human body or physical condition, that change affects the overall reflection intensity in the wavelength band but has substantially no effects on the ratio of the intensity at the particular wavelength to the average intensity in the wavelength band. Thus, chemical liquid injector 100 of the embodiment can determine the leak of the liquid with high accuracy to output and notify the alarm without being affected by variations in human body or physical condition, and the operator can immediately recognize removal of injection needle 230 from blood vessel V of the patient and takes measures.

In chemical liquid injector 100 of the embodiment, if the removal of injection needle 230 is detected as described above, the injection of the liquid can be automatically stopped to automatically prevent the liquid injection from being continued while injection needle 230 comes off blood vessel V. In addition, light-emitting diode 403 emits the rays in the wavelength band reflected favorably inside arm A and optical filter 406 allows only the rays in the wavelength band to pass therethrough to phototransistor 404, thereby making it possible to preclude erroneous detection of ambient light noise by phototransistor 404.

In chemical liquid injector 100 of the embodiment, leak-detecting unit 401 including light-emitting diode 403, phototransistor 404, wireless transmitting unit 409 and the like is formed separately from injection control unit 101 including wireless receiving unit 121, liquid crystal display 104, speaker unit 105 and the like.

When leak-detecting unit 401 detects the leak of the liquid, injection control unit 101 outputs and notifies the leak alarm through wireless communication. This structure can reduce the size and weight of leak-detecting unit 401 directly mounted on the human body to facilitate handling thereof and the leak alarm can be recognized without fail by the operator who is located away from leak-detecting unit 401 and manually operates injection control unit 101.

Injection control unit 101 continuously detects the reception state of the radio signal from leak-detecting unit 401 and outputs and notifies the reception state in real time. The operator can always know the communication state between leak-detecting unit 401 and injection control unit 101, and if the communication state is not favorable, the operator can deal with that problem before the injection operation is performed.

Since injection control unit 101 outputs and notifies the reception alarm when the detected reception state is degraded below the predetermined state, it is possible to prevent the situation in which the alarm signal cannot be wirelessly received due to poor communication and the leak alarm is not output. In addition, the liquid injection is stopped when at least one of the leak alarm and the reception alarm is output and notified, so that it is possible not only to automatically prevent the continuous liquid injection while injection needle 230 comes off blood vessel 501, but also to avoid the continuous liquid injection while the alarm signal cannot be wirelessly received.

The present inventor has experimentally checked whether or not a contrast medium leaked in a human body can be detected with rays. In the experiment, first, a light source capable of emitting rays at wavelengths of approximately 450 nm to 1350 nm was prepared, and the light source was placed at one end of a light-shield container and a spectrometer was disposed at the other end. Then, the intensity of rays emitted by the light source was measured by the spectrometer for each wavelength in the state where the container was filled with air as a reference, the state where the container was filled with water substituting for a human body, and the state where the container was filled with a typical contrast medium for CT. It was revealed that the contrast medium adequately absorbed the infrared rays at a wavelength near 950 nm as compared with the air and water.

Since a human body adequately passes infrared rays at wavelengths of approximately 800 to 1000 nm, the present inventor performed a second experiment by using an infrared LED (Light-Emitting Diode) with a peak wavelength of 950 nm as a light source. In the second experiment, a first chicken piece was placed at the bottom of a light-shield container and the container was sealed by a first transparent acrylic plate. A second acrylic plate was disposed in parallel with the first acrylic plate such that they are spaced from each other, and a second chicken piece was placed on the surface of the second acrylic plate. A model of a human body was formed in this manner (not shown).

An infrared LED with a peak wavelength of 950 nm and a spectrometer were placed in parallel to form a trial detecting unit (not shown) similar to leak-detecting unit 401 of the embodiment. The trial detecting unit was placed in intimate contact with the surface of the second chicken piece of the human body model. Then, the intensity of the infrared rays was measured by the spectrometer for each wavelength in the state where the human body model was filled with air between the first and second acrylic plates, the state where the model was filled with water, and the state where the model was filled a contrast medium. It was revealed that the contrast medium showed the detected intensity extremely reduced near at a wavelength of 950 nm as compared with the air and water.

In other words, in leak-detecting unit 401 of the embodiment, the infrared LED with a peak wavelength of 950 nm can be used as the light-emitting diode, the intensity of infrared rays at the wavelength of 950 nm can be detected by phototransistor 404, for example, thereby enabling favorable determination of a leak of the contrast medium. Since the infrared LED with a peak wavelength of 950 nm as described above is commercially available as a product, such a product can be used to realize leak-detecting unit 401 of the embodiment with high productivity.

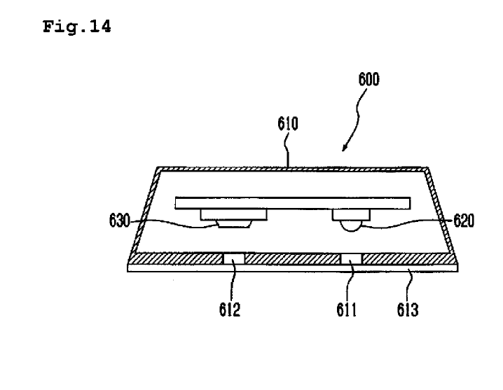

After seeing the foregoing results, the present inventor formed leak-detecting unit 600 by way of trial as shown in FIG. 14 to perform a third experiment. Leak-detecting unit 600 has unit housing 610 in a flat box shape. Unit housing 610 contains infrared LED 620 with a peak output wavelength of 950 nm and phototransistor 630 with a peak detection wavelength of 880 nm such that they face downward.

Unit housing 610 has circular opening holes 611 and 612 in a bottom plate at positions opposite to infrared LED 620 and phototransistor 630, respectively. Transparent sheet 613 made of resin such as PET (Polyethylene Terephthalate) and PS(Poly-Styrene) is placed over the entire bottom surface.

Enlarging and reducing opening hole 612 opposite to phototransistor 630 increases and reduces the amount of received light, but the effects of noise due to the surface state or the like of the human body are also increased and reduced, so that an extremely large or small size of opening hole 612 does not provide high detection sensitivity. The present inventor performed an experiment to see the detection sensitivity by setting the diameter of opening hole 612 to 1.0 mm, 1.5 mm, and 3.0 mm. The resulting S/N ratios (Signal-to-Noise ratio) were 1.8, 5.0, and 3.0, respectively. The maximum level was provided at the diameter of 1.5 mm.

In the environment where leak-detecting unit 600 as described above is used, a fluorescent lamp is typically used as illumination. A typical white fluorescent lamp hardly outputs rays at wavelengths of 800 μm or longer. As a result, it has been shown that leak-detecting unit 600 for detecting a liquid leak at a wavelength of 950 nm as described above can favorably function without being affected by outside light in a typical environment where the white fluorescent lamp is used as illumination.

### Modifications of the Embodiment

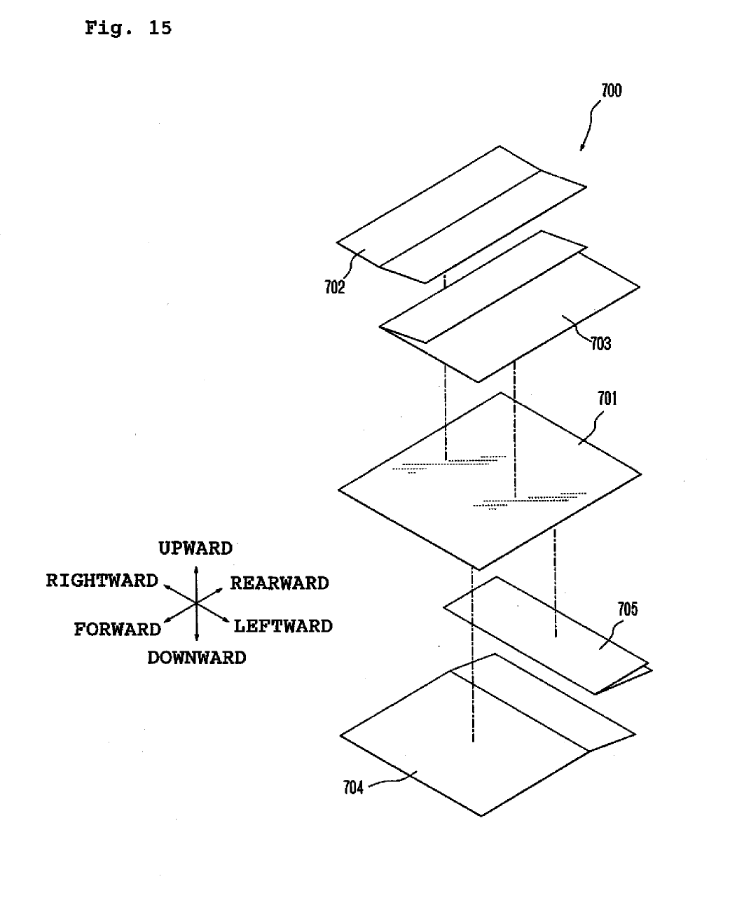

The present invention is not in any way limited to the abovementioned embodiment, but various changes and modifications may be made therein without departing from the scope of the invention. For example, in the above embodiment, peel sheets 510, 520, 530, and 540 of the same shape are affixed to sheet body 501 of the square shape in a symmetrical arrangement in the forward, rearward, leftward, and rightward directions. However, as in double-sided adhesive sheet 700 illustrated in FIG. 15, peel sheets 702 to 705 having different shapes may be affixed in an asymmetrical arrangement to sheet body 701 of a rectangle shape.

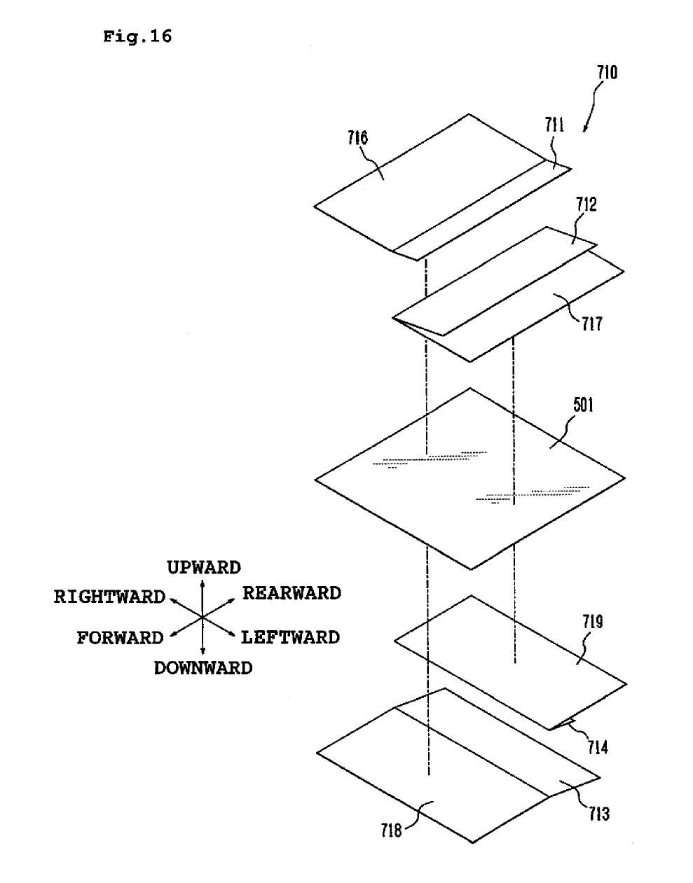

In the above embodiment, first, second, third, and fourth peeling portions 511, 521, 531, and 541 have the same shape. However, as in double-sided adhesive sheet 710 shown in FIG. 16, second and third peeling portions 721 and 713 may be formed to be larger than and extend off first and fourth peeling portions 711 and 714, respectively.

In double-sided adhesive sheet 710 described above, after first peeling portion 711 and second peeling portion 712 are overlapped on second affixing portion 717, first and second peeling portions 711 and 712 can be erected and held together easily, for example. In addition, after third peeling portion 713 and fourth peeling portion 714 are overlapped on fourth affixing portion 719, only third peeling portion 713 can be pulled easily to remove third affixing portion 718 from sheet body 501.

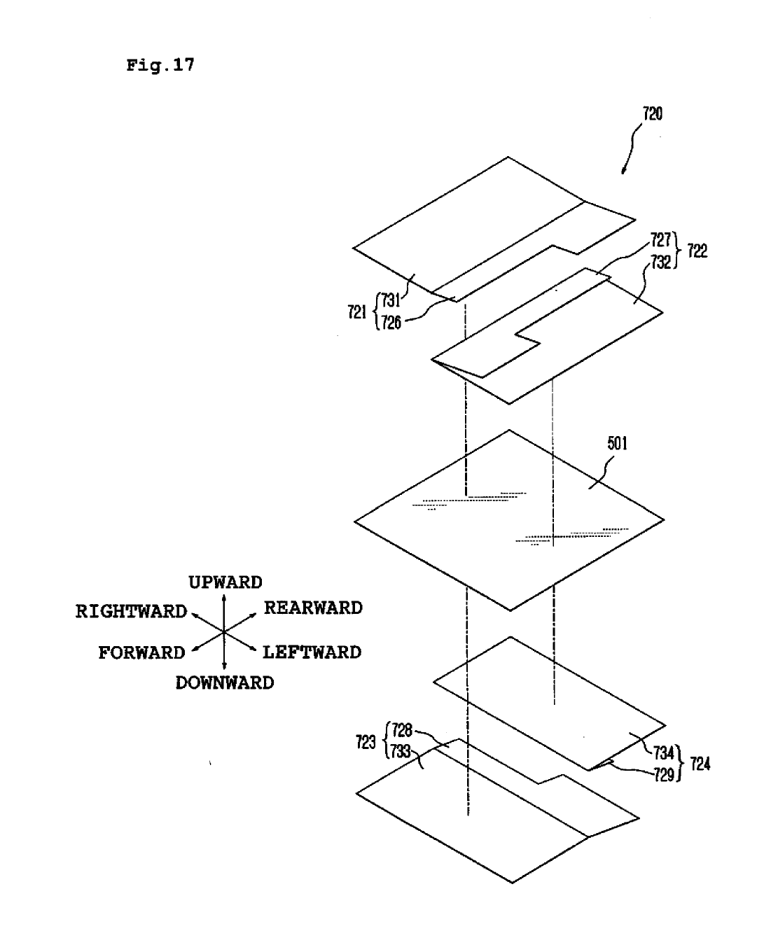

In the above embodiment, first, second, third, and fourth peel sheets 510, 520, 530, and 540 are formed in the rectangular shape. As in double-sided adhesive sheet 720 shown in FIG. 17, pealing portions 726, 727, 728, and 729 of first, second, third, and fourth peel sheets 721, 722, 723, and 724 may be formed in a stepped shape, for example.

In double-sided adhesive sheet 720, after first peeling portion 726 and second peeling portion 727 are overlapped on second affixing portion 732, first and second peeling portions 726 and 727 can be erected and held together easily, and this applies to third and fourth peeling portions 728 and 729.

In addition, any of first, second, third, and fourth peeling portions 726, 727, 728, and 729 can be selectively pulled to remove first, second, third, and fourth affixing portions 731, 732, 733, and 734 from sheet body 501, so that excellent handleability can be provided. Also, since first, second, third, and fourth peel sheets 721, 722, 723, and 724 are formed in the abovementioned stepped shape but have the same shape, the productivity of double-sided adhesive sheet 720 is excellent.

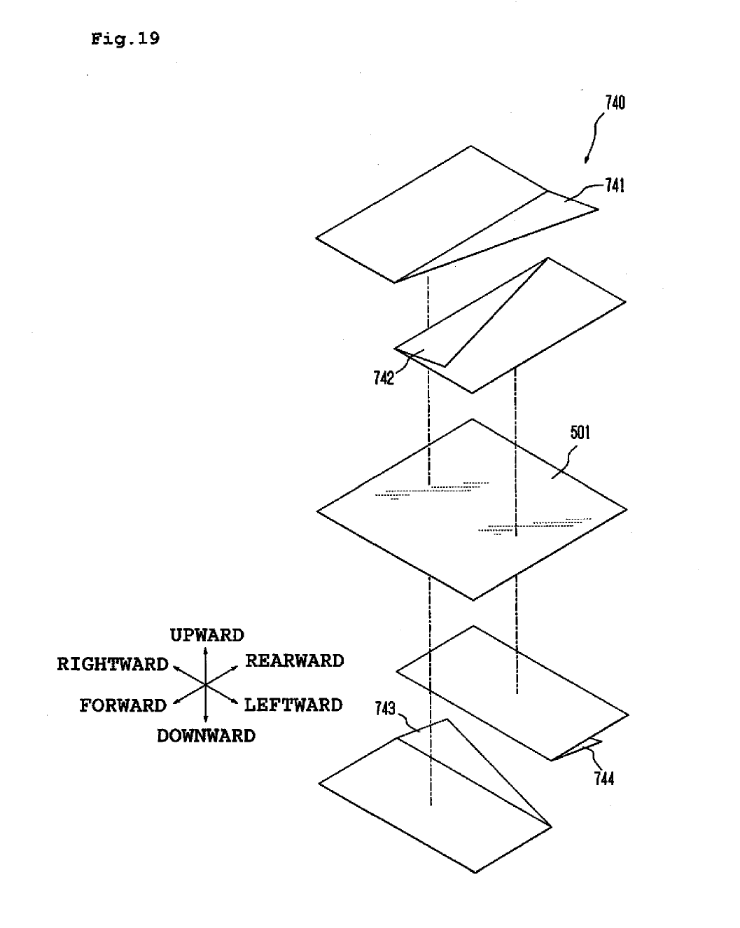

As shown by double-sided adhesive sheet 740 in FIG. 19, first, second, third, and fourth peeling portions 741, 742, 743, and 744 may be formed in a triangular shape. In

this case, excellent handleability can be provided similarly to double-sided adhesive sheet 720 described above. In addition, the simple outer shape of first, second, third, and fourth peeling portions 731, 742, 743, and 744 can further enhance the productivity.

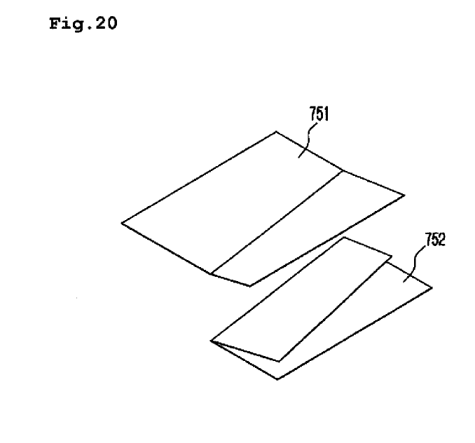

While first, second, third, and fourth peel sheets 510, 520, 530, and 540 are bent in parallel with the four sides of sheet body 501 in the above embodiment, they may be bent obliquely as shown by peel sheets 751 and 752 in FIG. 20.

In this case, handleability can also be improved similarly to double-sided adhesive sheets 720 and 740 described above. In addition, peel sheets 751 and 752 of the same rectangular shape as peel sheet 510 and the like described above may be used without any change, and bent peel sheets 751 and 752 have the same shape, thereby achieving favorable productivity.

Adhesive sheet body 501 is formed of the transparent resin sheet in the above embodiment. As shown in FIG. 21, it is possible that sheet body 760 can have image 761 representing the position of injection needle 230 to be held on the lower surface of sheet body 760 and image 762 representing the shape of leak-detecting unit 401 to be affixed to the upper surface, for example.

In use of sheet body 760 as described above, for example, an operator may strips third and fourth peel sheets 530 and 540 from sheet body 760 with first and second peel sheets 510 and 520 affixed thereto, and then affixes the exposed lower surface of sheet body 760 to the position of injection needle 230. This causes image 761 on the exposed lower surface of sheet body 760 to be matched to the position of injection needle 230.

Next, the operator strips first and second peel sheets 510 and 520 from the upper surface of sheet body 760 and affixes leak-detecting unit 401 thereto, thereby matching leak-detecting unit 401 to the position of image 762 on sheet body 760. In this manner, sheet body 760 can appropriately hold injection needle 230 and leak-detecting unit 401.

In sheet body 760 described above, it is essential only that image 761 representing the position of injection needle 230 should be delineated such that it can be identified visually from below and image 762 representing the shape of leak-detecting unit 401 should be delineated such that it can be identified visually from above.

For example, when sheet body 760 is transparent, the images may be printed on the upper surface or lower surface or may be formed with nontransparent resin through deposition or the like inside sheet body 760. On the other hand, when sheet body 760 is opaque, image 761 representing injection needles 230 may be delineated on the lower surface, image 762 representing leak-detecting unit 401 may be delineated on the upper surface, and the upward and downward directions may be clearly shown on peel sheet 510 or the like.

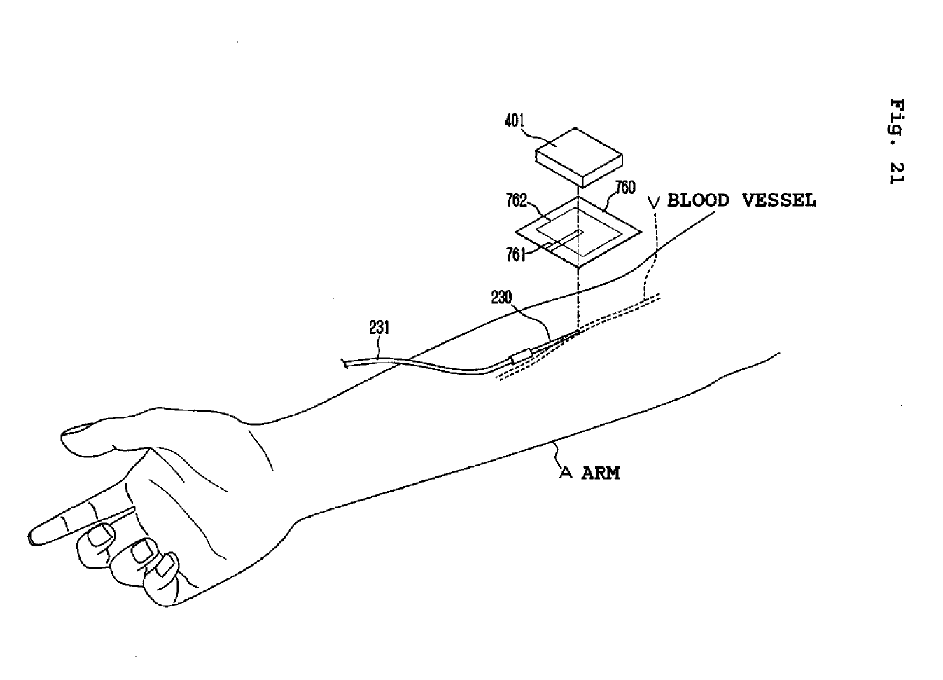

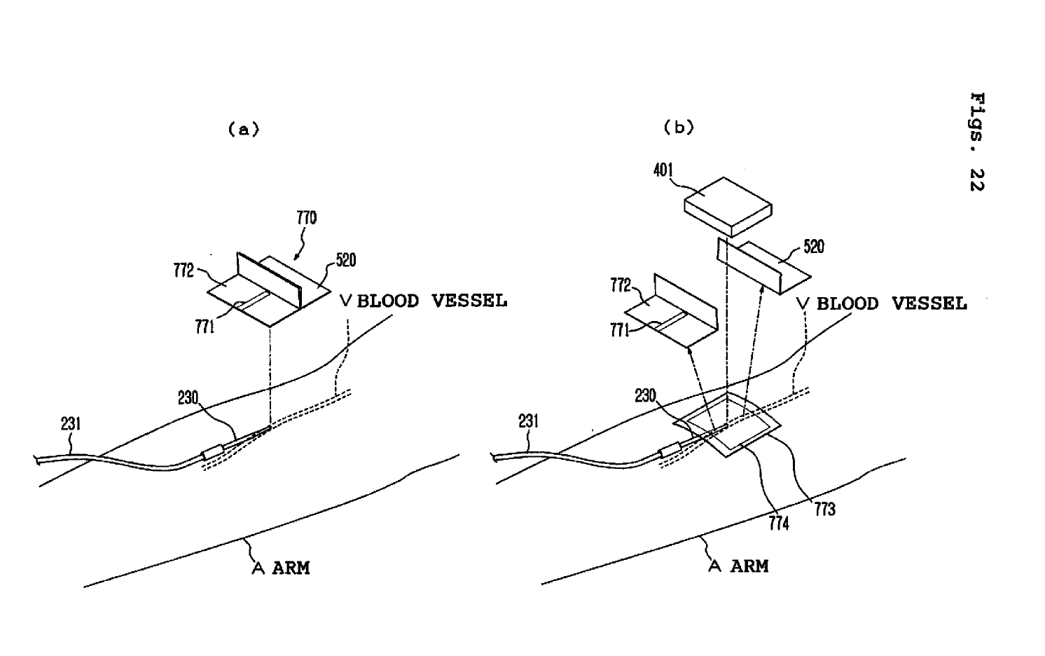

As shown by double-sided adhesive sheet 770 in FIG. 22, image 771 representing the position of injection needles 230 may be delineated on the upper surface of first peel sheet 772 and only image 774 representing the shape of leak-detecting unit 401 may be delineated on sheet body 773. In this case, double-sided adhesive sheet 770 can appropriately hold injection needles 230 and leak-detecting unit 401 more simply and reliably.

In the above embodiment, although sheet body 501 of double-sided adhesive sheet 500 favorably passes the rays in the predetermined wavelength band emitted and then received by leak-detecting unit 401, sheet body 500 can permit only the rays in the abovementioned wavelength band to pass therethrough.

More particularly, since a film-shaped optical filter for passing only rays in a predetermined wavelength band is commercially available (not shown), an adhesive may be applied to both sides of the optical filter to provide sheet body 501 as described above. In this case, sheet body 501 serves similarly to optical filter 406 and double-sided adhesive sheet 500 can improve the detection accuracy of leak-detecting unit 401.

In the above embodiment, the leak-detecting system is formed integrally with chemical liquid injector 100. The leak-detecting system may be formed separately from chemical liquid injector 100. Since the injection of the liquid must be stopped immediately after the leak of the liquid is detected as described above, the leak-detecting system formed integrally with chemical liquid injector 100 is effective. If the leak-detecting system is formed separately from chemical liquid injector 100, chemical liquid injector 100 preferably stops the injection operation in association with output of an alarm from the leak-detecting system.

In the above embodiment, chemical liquid injector 100 outputs and notifies the leak alarm and the reception alarm. For example, the alarm may be transmitted as data to control unit 302 of CT scanner 300 and then output and notified on liquid crystal display 304. Since control unit 302 is placed in a different room from imaging diagnostic unit 301 as described above, notifying imaging diagnostic unit 301 of the alarm is effective.

In the above embodiment, only chemical liquid injector 100 stops the injection operation in response to the leak alarm. For example, CT scanner 300 may also stop the imaging operation in association with the stop of the operation of chemical liquid injector 100 described above. In this case, chemical liquid injector 100 may directly transmit the alarm signal of leak-detecting unit 401 as data to CT scanner 300, or may indirectly transfer it as data via injection control unit 101.

The above embodiment assumes that chemical liquid injector 100 is used near CT scanner 300, but it may be used near a CT scanner, a PET apparatus, an angiography apparatus, an MRA apparatus, an ultrasonic diagnostic apparatus, or the like. In the above embodiment, leak-detecting unit 401 and injection control unit 101 wirelessly communicate with each other through radio signals. The communication method may be realized with wireless communication of ultrasonic signals, wireless communication of optical signals, wire communication of electric signals, wire communication of optical signals, or the like.

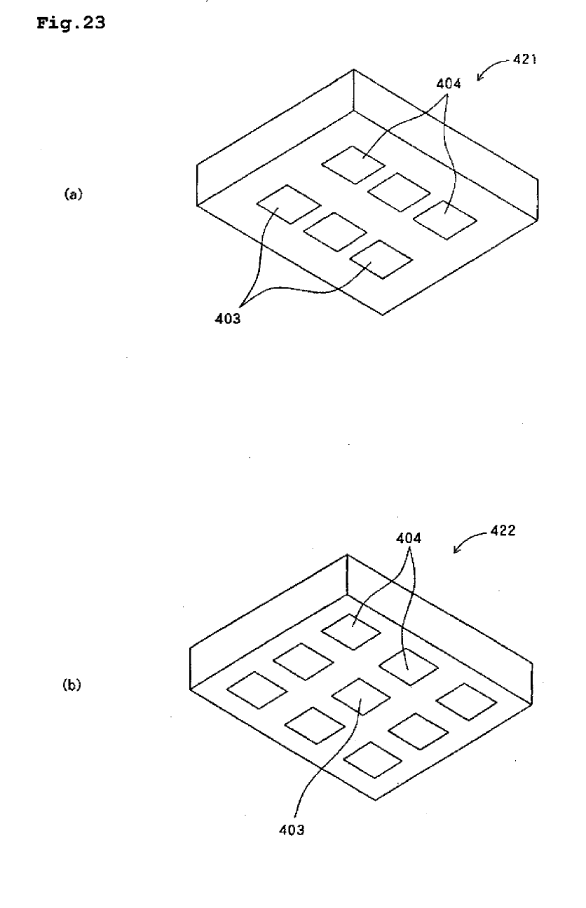

In the above embodiment, one light-emitting diode 403 and one phototransistor 404 are mounted on leak-detecting unit 401. Alternatively, it is possible to provide leak-detecting unit 421 which includes a plurality of light-emitting diodes 403 and a plurality of phototransistors 404 as shown in FIG. 23(a), or leak-detecting unit 422 which includes a plurality of phototransistors 404 placed around one light-emitting diode 403 as shown in FIG. 23(b). Since such a leak-detecting system can detect a leak of a liquid at a plurality of positions, the liquid leak can be detected more satisfactorily.

In the above embodiment, chemical liquid injector 100 causes leak alarming means 141 to output and notify only the leak alarm with an image displayed on liquid crystal display 104 or the like. For example, a graph displaying means (not shown) may be used to output and display a graph which represents distribution of intensity as shown in FIGS. 10(a) and 10(b) on liquid crystal display 104.

In the above embodiment, the only one particular wavelength is contained in the predetermined wavelength band of the rays, but a plurality of particular wavelengths may be used. In this case, the intensity can be measured for each of the plurality of particular wavelengths and the ratio can be calculated for each of the plurality of intensities. The occurrence of a leak may be determined when all of the plurality of ratios are higher than a reference value, for example. The occurrence of a leak is determined on the basis of the plurality of particular wavelengths in this case, so that false detection can be reduced to improve the accuracy of detection.

Alternatively, the occurrence of a leak may be determined when one of the plurality of ratios is higher than the reference value, thereby preventing a failure to detect a liquid leak. In addition, both of the failure to detect liquid leak and the false detection may be prevented favorably by determining the occurrence of a leak if a predetermined number of the plurality of ratios are higher than the reference value or if the majority of the plurality of ratios are higher than the reference value. When the predetermined wavelength band of the rays contains the plurality of particular wavelengths as described above, the plurality of calculated ratios may be individually compared with specific reference values to improve the accuracy of determination of occurrence of a leak.

In the above embodiment, light-emitting diode 403 emits the rays in the predetermined wavelength band containing the particular wavelength at which the reflectivity for the liquid is lower than the reflectivity for the internal tissues of arm A, and the occurrence of a leak is determined if the intensity of the rays at the particular wavelength reflected inside arm A is lower than the average intensity in the wavelength band.

Alternatively, light-emitting diode 403 may emit rays in a wavelength band containing a particular wavelength at which the reflectivity for the liquid is higher than the reflectivity for the internal tissues of arm A, and the occurrence of a leak is determined if the ratio of the intensity of the rays at the particular wavelength to the average intensity in the wavelength band reflected inside arm A is higher than a reference value.

It is also possible that light-emitting diode 403 can emit rays containing a first particular wavelength at which the reflectivity for the liquid is higher than the reflectivity for the internal tissues of arm A and a second particular wavelength at which the reflectivity for the liquid is equal to the reflectivity for the internal tissues, and that the occurrence of a leak can be determined if the intensity of the reflected rays at the first particular wavelength to the intensity of the reflected rays at the second particular wavelength is higher than a reference vale.

It is also possible that light-emitting diode 403 can emit rays containing a first particular wavelength at which the reflectivity for the liquid is lower than the reflectivity for the internal tissues of arm A and a second particular wavelength at which the reflectivity for the liquid is equal to the reflectivity for the internal tissues, and that the occurrence of a leak can be determined if the intensity of the reflected rays at the first particular wavelength to the intensity of the reflected rays at the second particular wavelength is lower than a reference vale.

It is also possible that light-emitting diode 403 can emit rays containing a first particular wavelength at which the reflectivity for the liquid is higher than the reflectivity for the internal tissues of arm A and a second particular wavelength at which the reflectivity for the liquid is lower than the reflectivity for the internal tissues, and that the occurrence of a leak can be determined if the intensity of the reflected rays at the first particular wavelength to the intensity of the reflected rays at the second particular wavelength is higher than a reference vale.

In the structure in which the wavelength band contains the first and second particular wavelengths as described above, it is possible that only the first particular wavelength can be realized by a plurality of wavelengths, and that the ratios of the intensities of the reflected rays at the plurality of first particular wavelengths to the intensity of the reflected rays at the second particular wavelength can be individually compared with a plurality of reference values. It is also possible that only the second particular wavelength can be realized by a plurality of wavelengths, and that the ratios of the intensity of the reflected rays at the first particular wavelength to the intensities of the reflected rays at the plurality of second particular wavelengths can be individually compared with a plurality of reference values. Alternatively, each of the first and second particular wavelengths can be realized by a plurality of wavelengths, and the ratios of the intensities of the reflected rays at the plurality of first wavelengths to the intensities of the rays at the plurality of second particular wavelengths can be individually compared with a plurality of reference values.

In the above embodiment, when the leak of the liquid is detected, chemical liquid injector 100 stops piston driving mechanism 116 to discontinue the liquid injection. For example, the leak-detecting system may include a standalone tube blocking means (not shown) for blocking the flow of the liquid in extension tube 231 when a leak of the liquid is detected.

Such a tube blocking means is formed as a standalone unit structure which is mounted on extension tube 231, and performs wireless or wire communication with leak-detecting unit 401 and the detecting apparatus body, for example. The tube blocking means also includes a mechanism for opening or closing extension tube 231 with a driving source such as a solenoid, and blocks extension tube 231 when the leak of the liquid is detected. In such a leak-detecting system, extension tube 231 is blocked independently when the liquid leak is detected, so that the liquid injection can be stopped automatically even when chemical liquid injector 100 does not operate in association with the leak-detecting system, for example.

In the above embodiment, microprocessor 130 functions in accordance with the installed computer program to logically realize various means 141 to 146 of injection control unit 101. For example, at least some of various means 141 to 144 may be formed by hardware such as a dedicated logical circuit.

In contrast, in the above embodiment, various means 411 to 414 of leak-detecting unit 401 are formed by the predetermined hardware, but various means 411 to 414 may be logically realized by a microprocessor which functions in accordance with an installed computer program, for example.

In the above embodiment, one liquid syringe 200 is mounted in one concave portion 114 of chemical liquid injector 100, but a plurality of liquid syringes 200 may be mounted in a plurality of concave portions of the injection head (not shown). In the above embodiment, liquid syringe 200 is directly mounted on chemical liquid injector 100. Since liquid syringes 200 of various sizes are commercially available, it is possible that only liquid syringe 200 of the maximum size can be directly mounted on chemical liquid injector 100 and liquid syringes 200 of the various sizes other than the maximum size can be mounted on chemical liquid injector 100 with dedicated cylinder adapters (not shown) interposed between them.

1. A double-sided adhesive sheet comprising:
a sheet body including an upper surface and a lower surface, each of the surfaces having adhesion;
a first peel sheet having a first affixing portion affixed to a portion of the upper surface of the sheet body, and a first peeling portion not affixed to the upper surface;
a second peel sheet having a second affixing portion affixed to the upper surface of the sheet body, and a second peeling portion not affixed to the upper surface, said first and second peeling portions overlap each other;
a third peel sheet having a third affixing portion affixed to the lower surface of the sheet body, and a third peeling portion not affixed to the lower surface; and
a fourth peel sheet having a fourth affixing portion affixed to the lower surface of the sheet body, and a fourth peeling portion not affixed to the lower surface, said third and fourth peeling portions overlap each other,
wherein a boundary between the first affixing portion and the second affixing portion intersects a boundary between the third affixing portion and the fourth affixing portion.

2. The double-sided adhesive sheet according to claim 1, wherein the sheet body is rectangular shape,
the first affixing portion is affixed to a left half of the upper surface of the sheet body;
the second affixing portion is affixed to a right half of the upper surface of the sheet body;
the third affixing portion is affixed to a forward half of the lower surface of the sheet body; and
the fourth affixing portion is affixed to rearward half of the lower surface of the sheet body.

3. The double-sided adhesive sheet according to claim 1, wherein all the peel sheets are the same shape.

4. The double-sided adhesive sheet according to claim 1, wherein one of said peeling portion extends partially with respect to the other peeling portion on the same side.

5. (canceled)

6. (canceled)

7. The double-sided adhesive sheet according to claim 1, wherein the sheet body has an image formed thereon such that the image is visually identified from below, the image representing a position of an object to be held on the lower surface, and
the sheet body has an image formed thereon such that the image is visually identified from above, the image representing a shape of an object to be affixed to the upper surface.

8. The double-sided adhesive sheet according to claim 1, wherein at least one of the first affixing portion and the second affixing portion has an image formed thereon such that the image is visually identified from above, the image representing a position of an object to be held on the lower surface of the sheet body, and
the sheet body has an image formed thereon such that the image is visually identified from above, the image representing a shape of an object to be affixed to the upper surface.

9. (canceled)

10. (canceled)

11. A leak-detecting system for detecting a leak of a liquid injected into a blood vessel of a human body near a surface thereof through an injection needle, comprising:
a leak-detecting unit for detecting the leak and wirelessly transmitting data indicating the leak;
a detecting apparatus body for outputting and notifying the leak wirelessly transmitted from the leak-detecting unit; and
the double-sided adhesive sheet for affixing the leak-detecting unit to the surface of the human body with the sheet body according to claim 1.

12. (canceled)

13. (canceled)

14. (canceled)

15. A chemical liquid injector comprising:
a piston-driving mechanism for pressing a piston member of a syringe;
the leak-detecting system according to claim 11; and
an input stopping means for stopping injection of the liquid when the leak-detecting system outputs and notifies the leak.

16. The double-sided adhesive sheet according to claim 8, wherein the image indicates a position for an injection needle.

17. The double-sided adhesive sheet according to claim 1, wherein the sheet body serves as an optical filter.
